(19) United States
(12) Patent Application Publication
(10) Pub. No.: US 2005/0107681 A1
(43) Pub. Date: May 19, 2005

(54) Wireless patient monitoring device for magnetic resonance imaging

(76) Inventor: David M. Griffiths, Pittsburgh, PA (US)

Correspondence Address: GREGORY L BRADLEY;MEDRAD INC, ONE MEDRAD DRIVE, INDIANOLA, PA, 15051, US

(21) Appl. No.: 10/897,737

(22) Filed: Jul. 23, 2004

Related U.S. Application Data
(60) Provisional application No. 60/489,592, filed on Jul. 23, 2003.

(51) Int. Cl. A61B 5/05

(52) U.S. Cl. 600/410

(57) ABSTRACT

The invention relates to systems, methods, and associated devices for wirelessly communicating physiologic signals or other data in an electromagnetically noisy environment, such as a magnetic resonance imaging (MRI) suite. They permit wireless communication of data obtained from a sensor module attached to a patient while situated within the bore of an MR scanner. The system includes a first transceiver and a second transceiver. The first transceiver is linked to the sensor module for transmitting the data received therefrom. The second transceiver, which is connected to an apparatus remote from the first transceiver, is used to convey to the apparatus the data received from the first transceiver. The first and second transceivers enable the sensor module and the apparatus to communicate unidirectionally or bidirectionally without being adversely affected by, or adversely affecting, the operation of the MR system.

### CROSS-REFERENCE TO RELATED APPLICATION

This application for patent claims the benefit of U.S. Provisional Application Ser. No. 60/489,592 titled Wireless Patient Monitor Device For Magnetic Resonance Imaging, filed 23 Jul. 2003. This provisional application has been assigned to the assignee of the invention disclosed below, and its teachings are incorporated into this document by reference.

### FIELD OF THE INVENTION

The invention relates generally to systems and methods of communication for use during magnetic resonance (MR) imaging and spectroscopy procedures. More particularly, the invention relates to wireless communication between and/or within the various rooms of an MR suite. Even more particularly, the invention pertains to systems and methods, and associated devices therefor, for wirelessly communicating physiologic data between the patient in the bore of an MR scanner and the monitoring equipment therefor located elsewhere in the MR suite.

### BRIEF DESCRIPTION OF RELATED ART

The following information is provided to assist the reader to understand the invention disclosed below and the environment in which it will typically be used.

Magnetic resonance imaging (MRI) is a noninvasive method of producing high quality images of the interior of the human body. It allows medical personnel to see inside the body (e.g., organs, muscles, nerves, bones, and other structures) without surgery or the use of potentially harmful ionizing radiation such as X-rays. The images are of such high resolution that disease and other pathological conditions can often be visually distinguished from healthy tissue. Magnetic resonance (MR) systems and techniques have also been developed for performing spectroscopic analyses by which the chemical content of tissue or other material can be ascertained.

MR imaging and spectroscopic procedures are performed in what is known as an MR suite. As shown in FIG. 1A, an MR suite typically has three rooms: a scanner room 1, a control room 2, and an equipment room 3. The scanner room 1 houses the MR scanner 10 into which a patient is moved via a slideable table 11 to undergo a scanning procedure, and the control room 2 contains a computer console 20 from which the operator controls the overall operation of the MR system. In addition to a door 4, a window 5 is typically set in the wall separating the scanner and control rooms to allow the operator to observe the patient during such procedures. The equipment room 3 contains the various subsystems necessary to operate the MR system. The equipment includes a power gradient controller 31, a radio frequency (RF) assembly 32, a spectrometer 33, and a cooling subsystem 34 with which to avoid the build up of heat which, if left unaddressed, could otherwise interfere with the overall performance of the MR system. These subsystems are typically housed in separate cabinets, and are supplied electricity through a power distribution panel 12 as are the scanner 10 and the slideable patient table 11.

An MR system obtains such detailed images and spectroscopic results by taking advantage of a basic property of the hydrogen atom, which is found in abundance in all cells within the body. Within the cells, the nuclei of hydrogen atoms naturally spin like a top, or precess, randomly in every direction. When subject to a strong magnetic field, however, the spin-axes of the hydrogen nuclei align typically themselves in the direction of that field. This is because the nucleus of the hydrogen atom has what is referred to as a large magnetic moment, which is basically an inherent tendency to line up with the direction of the magnetic field to which it is exposed. During an MR scan, the body or a region thereof is exposed to such a magnetic field. This causes the hydrogen nuclei of the exposed region(s) to line up—and collectively form an average vector of magnetization—in the direction of that magnetic field.

As shown in FIGS. 1B and 1C, the scanner 10 is comprised of a main magnet 101, three gradient coils 103a-c, and, usually, an RF antenna 104 (often referred to as the whole body coil). Superconducting in nature, the main magnet 101 is typically cylindrical in shape. Within its cylindrical bore, the main magnet 101 generates a strong magnetic field, often referred to as the Bo or main magnetic field, which is both uniform and static (non-varying). For a scanning procedure to be performed, the patient must be moved into this cylindrical bore, typically while supine on table 11, as best shown in FIGS. 1B and 1C. The main magnetic field is oriented along the longitudinal axis of the bore, referred to as the z direction, which compels the magnetization vectors of the hydrogen nuclei in the body to align themselves in that direction. In this alignment, the hydrogen nuclei are prepared to receive RF energy of the appropriate frequency from RF coil 104. This frequency is known as the Larmor frequency and is governed by the equation ω=γB₀, where ω is the Larmor frequency (at which the hydrogen atoms precess), γ is the gyromagnetic constant, and B₀ is the strength of the main magnetic field.

The RF coil 104 is typically used both to transmit pulses of RF energy and to receive the resulting magnetic resonance (MR) signals induced thereby in the hydrogen nuclei. Specifically, during its transmit cycle, the coil 104 broadcasts RF energy into the cylindrical bore. This RF energy creates a radio frequency magnetic field, also known as the RF B₁ field, whose magnetic field lines point in a direction perpendicular to the magnetization vectors of the hydrogen nuclei. The RF pulse (or B₁ field) causes the spin-axes of the hydrogen nuclei to tilt with respect to the main (B₀) magnetic field, thus causing the net magnetization vectors to deviate from the z direction by a certain angle. The RF pulse, however, will affect only those hydrogen nuclei that are precessing about their axes at the frequency of the RF pulse. In other words, only the nuclei that “resonate” at that frequency will be affected, and such resonance is achieved in conjunction with the operation of the three gradient coils 103a-c.

Each of the three gradient coils is used to vary the main (B₀) magnetic field linearly along only one of the three spatial directions (x,y,z) within the cylindrical bore. Positioned inside the main magnet as shown in FIG. 1C, the gradient coils 103a-c are able to alter the main magnetic field on a very local level when they are turned on and off very rapidly. Thus, in conjunction with the main magnet 101, the gradient coils can be operated according to various imaging techniques so that the hydrogen nuclei—at any given point or in any given strip, slice or unit of volume—will be able to achieve resonance when an RF pulse of the appropriate frequency is applied. In response to the RF pulse, the precessing hydrogen nuclei in the selected region absorb the RF energy being transmitted from RF coil 104, thus forcing the magnetization vectors thereof to tilt away from the direction of the main (B₀) magnetic field. When the RF coil 104 is turned off, the hydrogen nuclei begin to release the RF energy they just absorbed in the form of magnetic resonance (MR) signals, as explained further below.

One well known technique that can be used to obtain images is referred to as the spin echo imaging technique. Operating according to this MR sequence, the MR system first activates one gradient coil 103a to set up a magnetic field gradient along the z-axis. This is called the “slice select gradient,” and it is set up when the RF pulse is applied and is shut off when the RF pulse is turned off. It allows resonance to occur only within those hydrogen nuclei located within a slice of the region being imaged. No resonance will occur in any tissue located on either side of the plane of interest. Immediately after the RF pulse ceases, all of the nuclei in the activated slice are “in phase,” i.e., their magnetization vectors all point in the same direction. Left to their own devices, the net magnetization vectors of all the hydrogen nuclei in the slice would relax, thus realigning with the z direction. Instead, however, the second gradient coil 103b is briefly activated to create a magnetic field gradient along the y-axis. This is called the “phase encoding gradient.” It causes the magnetization vectors of the nuclei within the slice to point, as one moves between the weakest and strongest ends of this gradient, in increasingly different directions. Next, after the RF pulse, slice select gradient and phase encoding gradient have been turned off, the third gradient coil 103c is briefly activated to create a gradient along the x-axis. This is called the “frequency encoding gradient” or “read out gradient,” as it is only applied when the MR signal is ultimately measured. It causes the relaxing magnetization vectors to be differentially re-excited, so that the nuclei near the low end of that gradient begin to precess at a faster rate, and those at the high end pick up even more speed. When these nuclei relax again, the fastest ones (those which were at the high end of the gradient) will emit the highest frequency of radio waves and the slowest ones emit the lowest frequencies.

The gradient coils 103a-c therefore spatially encode these radio waves, so that each portion of the region being imaged is uniquely defined by the frequency and phase of its resonance signal. In particular, as the hydrogen nuclei relax, each becomes a miniature radio transmitter, giving out a characteristic pulse that changes over time, depending on the local microenvironment in which it resides. For example, hydrogen nuclei in fats have a different microenvironment than do those in water, and thus emit different pulses. Due to these differences, in conjunction with the different water-to-fat ratios of different tissues, different tissues emit radio signals of different frequencies. During its receive cycle, RF coil 104 detects these miniature radio emissions, which are often collectively referred to as the MR signal(s). From the RF coil 104, these unique resonance signals are conveyed to the receivers of the MR system where they are converted into mathematical data. The entire procedure must be repeated multiple times to form an image with a good signal-to-noise ratio (SNR). Using multidimensional Fourier transformations, the MR system then converts the mathematical data into a two- or even a three-dimensional image of the body, or region thereof, that was scanned.

As shown partially in FIGS. 1A and 1C, the scanner room 1 is shielded to prevent the entry and exit of electromagnetic waves. Specifically, the materials and design of its ceiling, floor, walls, door, and window effectively form a barrier or shield 6 that prevents the electromagnetic signals generated during a scanning procedure (e.g., the RF energy) from leaking out of scanner room 1. Likewise, shield 6 is designed to prevent external electromagnetic noise from leaking into the scanner room 1. The shield 6 is typically composed of a copper sheet material or some other suitable conductive layer. The window 5, however, is typically formed by sandwiching a wire mesh material between sheets of glass or by coating the window with a thin layer of conductive material to maintain the continuity of the shield. The conductive layer also extends to the door 4, which when open allows access to the scanner room 1 and yet when closed is grounded to and constitutes a part of shield 6. The ceiling, floor, door and walls of shield 6 provide approximately 100 decibels (dB) of attenuation, and window 5 approximately 80 dB, for the typical operating range of MR scanners (˜20 to 200 MHz). Barrier 6 thus shields the critical components (e.g., scanner, preamplifiers, receivers, local coils, etc.) of the MR system from undesirable sources of electromagnetic radiation (e.g., radio signals, television signals, and other electromagnetic noise present in the local environment).

The shield 6 serves to prevent external electromagnetic noise from interfering with the operation of the scanner 10, which if not addressed could otherwise result in degradation of the images and/or spectroscopic results obtained during a scanning procedure. For the scanner 10 to operate, however, the shield 6 must still allow communication of data and control signals between the scanner room 1 and the control and equipment rooms 2 and 3, and such communication is generally accomplished through a penetration panel 16.

As shown in FIG. 1A, the penetration panel 16 is typically incorporated into the wall between the scanner and equipment rooms 1 and 3. It features several ports through which the scanner 10 and other devices in the scanner room 1 are connected by cables to the computer console 20 and control subsystems in the control and equipment rooms 2 and 3, respectively. Each port typically includes a filtered BNC connector, which allows the communication of data and/or control signals while still maintaining the barrier to unwanted electromagnetic signals.

As is well known, several auxiliary systems have been designed for use in the MR suite, some of which requiring communication across the isolation barrier. These auxiliary systems are typically bifurcated, i.e., they have two pieces of equipment, with one piece located in the scanner room and the other situated in the control room. Some MR suites offer, or were retrofitted with, penetration panels with additional ports, which spawned the development of bifurcated systems that took advantage of this added functionality. In such auxiliary systems, the two pieces of equipment on opposite sides of the shield are hardwired through such a port via RF cables with the appropriate connectors. The ports are tuned and filtered to prevent transmission of frequencies therethrough that could adversely affect the operation of the MR system. The RF cables are similarly shielded, grounded and filtered to ensure that no external noise is coupled into the scanner room and thus defeat the purpose of the isolation barrier.

Other auxiliary systems employ different ways of communicating across the electromagnetic shield. The bifurcated injector system disclosed in U.S. Pat. No. 5,494,036 to Uber, III et al., incorporated herein by reference, is one such example. It allows contrast media to be injected into the blood stream of a patient undergoing an MR procedure. (Contrast media serves to increase the contrast between the different types of tissues in the region of the body undergoing a scan, and thereby enhance the resolution of the images obtained during the scanning procedure.) In this bifurcated system, an injector control unit in the scanner room with which to inject the contrast media into the patient communicates with a controller therefor situated in the control room. The '036 patent discloses that the injection control unit and its controller communicate across the barrier by either a dedicated fiber optic link or a pair of matched transceivers. In the preferred embodiment, the transceivers are attached to, and aimed at each other through, opposite sides of the window. They allow the injection control unit and controller to communicate with each other at frequencies that readily penetrate the shield yet do not adversely affect the operation of the MR system, preferably at wavelengths in either the infrared or visual portions of the electromagnetic spectrum. The injection control unit is itself typically shielded, and any spurious electromagnetic noise generated by the controller is shielded from the scanner by virtue of its isolation in the control room.

U.S. Patent Application Publication 2003/0058502 A1, incorporated herein by reference, discloses a system of wirelessly communicating across the isolation barrier between the two transceivers of a bifurcated equipment system, such as an injection system. The disclosed communication system is manifested as an antenna coupling having two antennas, one of which for communicating with the transceiver (for the injection control unit) on one side of the barrier and the other antenna for communicating with the transceiver (for the controller) on the other side of the barrier.

Although the '036 patent and related art constitute an advance over earlier communications systems targeted for the MR environment, there is still a need to develop communication systems that overcome the disadvantages inherent to such art. One disadvantage of the system disclosed in the '036 patent is that the cables used to connect to the transceivers on either side of the window inevitably restrict the mobility of the equipment in both the scanner and control rooms. Although the communication system disclosed in the published application enables mobility of the equipment on either side of the barrier, the two antennas of its antenna coupling are physically interconnected. Another shortcoming is that its antenna coupling is limited to permitting communication across the barrier, and thus does not contemplate the need for intra-room communication of data or other signals in the MR suite.

Monitoring the vital functions of patients is becoming increasingly more common in the MR environment. Examples of the physiologic functions that are commonly monitored include the oxygen saturation of arterial blood using pulse oximetry and the electrical activity of the brain via an electroencephalograph (EEG). Other electrophysiological signals that can be monitored include electro-oculograms (EOGs), electroencephalograms (EEGs), and electromyograms (EMGs). Respiration and blood pressure are two other physiologic parameters that are routinely monitored, as is the electrical activity of the heart by way of an electrocardiograph (ECG).

The heart is primary composed of muscle tissue that contracts and relaxes rhythmically to propel blood through the circulatory system of the body. The heartbeat begins with a small nerve bundle located in the upper right-hand corner of the right atrium, an area known as the sinoatrial (SA) node or pacemaker. Cells in the SA node generate electrical impulses at regular intervals of about 60-70 times per minute, though that rate can be increased or decreased by nerves external to the heart that respond to the physiologic demands of the body as well as to other (chemical) stimuli. These impulses travel through and synchronize the rest of the heart, and initiate the depolarization and subsequent repolarization of its muscle, thus causing the heart to contract and relax with a regular, steady beat. This depolarization is distributed from cell to cell, in the form of a wave through the muscle and certain nerve fibers of the heart. Once depolarization is complete, the cardiac cells are able to restore their resting polarity through a process called repolarization. The electrical activity of the heart can be detected through the conductive tissues of the body at the surface by electrodes applied to the skin. A small amount of conductive gel is often applied to the skin, which allows these signals to be more easily transmitted to the electrodes. Each electrode typically has a metallic detent or connective point to which an electrically conductive leadwire is attached by a corresponding clip. Each leadwire carries a bioelectric signal voltage from its corresponding electrode to an instrument known as an electrocardiograph or to other suitable monitoring equipment. The resulting cardiac signal is derived from the difference in voltages measured as a function of time between two such electrodes. The cardiac signal appears as peaks and valleys in a graphic image known as an electrocardiogram or signal (ECG). For basic ECG monitoring, a 3-lead lead-set is typically used. Lead-sets with a greater number of leads/electrodes can be used, however, if more detail (e.g., as to the different phases of the heartbeat) is needed to increase the likelihood of detecting a wider range of cardiac anomalies.

Physiologic monitoring in the MR suite, however, is complicated by the electromagnetic environment within the scanner room. This is because electrically conductive wires are typically used to convey physiologic data in the form of signal voltages from the patient to the monitoring equipment. The RF pulses and the varying magnetic fields generated during an MR scan tend to induce spurious electrical noise in such wires, with the noise appearing as artifacts in the signal voltages. Electronic devices commonly found in MR suites, such as fans and lights, may also emit electromagnetic emanations that can induce noise in the wires. In addition, any movement of the wires in the magnetic fields also tends to induce artifacts in the signal voltages. Besides these noise and motion artifacts, the RF pulses from the scanner, depending on their strength, can induce currents of a magnitude sufficient to cause heating of the wires, which can potentially subject the patient to the risk of burn.

The 9500 Multi-Gas Monitor, produced by Medrad, Inc., of Indianola, Pa., uses fiber optic links for communicating ECG and pulse oximetry data between a sensor device connected to the patient in the bore of the scanner and the monitor located elsewhere in the MR suite, often in the scanner room. This is disclosed in U.S. Pat. No. 6,052,614 to Morris, Sr. et al., incorporated herein by reference. The fiber optic cable provides a degree of immunity from noise and motion artifacts and also reduces the amount of noise radiated by the monitoring equipment that could adversely affect the images produced by the MR system. The fiber optic cable also isolates the patient from the RF energy produced by the scanner, and thus eliminates the risk of burn or shocks that could otherwise occur through the use of electrically conductive cables. The disadvantages of this system, as with the cable-dependent devices cited above, are that the fiber optic cable not only poses an obstacle to the operator in the scanner room but also limits the mobility and placement of the monitor within the MR suite.

In addition, there are some bifurcated systems, such as Magnitude™ patient monitor from Invivo Research, Inc., and injection systems from Medtron Medical Systems, Inc. (Saarbrücken, Germany), whose communication systems employ RF signals at high frequencies to penetrate the shield thereby permitting data to be conveyed between the scanner and control rooms. These products, however, still rely on cables that connect a sensor device on the patient in the bore of the scanner and the monitor therefor situated outside the bore. It would therefore be desirable to have a wireless connection between the sensor device (located within the bore) and the monitoring equipment therefor (located within the scanner or control rooms). Such a wireless connection could also be used to couple signals from the patient sensor device to the MR system, (e.g., ECG signals, which can be used to trigger operation of the scanner for acquiring images of the heart at the appropriate point during the cardiac cycle).

### SUMMARY OF THE INVENTION

Several objectives and advantages of the invention are attained by the various embodiments and related aspects of the invention summarized below.

In a presently preferred embodiment, the invention provides a system for wirelessly communicating physiologic data indicative of the condition of a patient exposed to a scanner of an MR system. The system includes a sensor mechanism, a first transducer circuit, a first RF transceiver circuit, a second RF transceiver circuit, and a second transducer circuit. The sensor mechanism is used to acquire the physiologic data from the patient. The first transducer circuit connects to the sensor mechanism for converting the physiologic data received therefrom from optical format to electrical format. The first RF transceiver circuit is connected to the first transducer circuit for transmitting the physiologic data received therefrom. The second RF transceiver circuit, which is remote from the first RF transceiver circuit, is used to receive the physiologic data transmitted by the first RF transceiver circuit. The second transducer circuit is connected to the second RF transceiver circuit for converting the physiologic data received therefrom from electrical format to optical format, and for conveying the physiologic data to an apparatus remote from the sensor mechanism. The communication between the sensor mechanism and the apparatus via the first and second RF transceiver circuits is accomplished without adversely affecting, or being adversely affected by, operation of the MR system.

In a related embodiment, the invention provides a system for wirelessly communicating data in an electromagnetically noisy environment. The system includes a first transducer circuit, a first RF transceiver circuit, a second RF transceiver circuit, and a second transducer circuit. The first transducer circuit connects to a first device of a bifurcated system for converting the data received therefrom from optical format to electrical format. The first RF transceiver circuit is connected to the first transducer circuit for transmitting the data received therefrom. The second RF transceiver circuit, which is remote from the first RF transceiver circuit, is used to receive the data transmitted by the first RF transceiver circuit. The second transducer circuit is connected to the second RF transceiver circuit for converting the data received therefrom from electrical format to optical format, and for conveying the data to a second device of the bifurcated system. The scheme of communication employed by the first and second RF transceivers enables the first and second devices to communicate without being adversely affected by noise in the environment.

In another related embodiment, the invention provides a system for wirelessly communicating data in an MR suite. The system includes a first transceiver circuit and a second transceiver circuit. The first transceiver circuit connects to a sensor module for transmitting the data received therefrom and for conveying to the sensor module the data transmitted thereto. The second transceiver circuit, which is connected to a monitoring apparatus, is used to convey the data received from the first transceiver circuit to the monitoring apparatus and to transmit to the first transceiver circuit the data received from the monitoring apparatus. The first and second transceiver circuits communicate using predetermined frequencies outside a range of, and without adversely affecting, operation of equipment situated in the MR suite.

In a different embodiment, the invention provides a system for wirelessly communicating data obtained from a sensor module attached to a patient situated within an imaging scanner. The system includes a first transceiver and a second transceiver. The first transceiver is linked to the sensor module for transmitting the data received therefrom. The second transceiver, which is connected to an apparatus remote from the first transceiver, is used to convey to the apparatus the data received from the first transceiver. The first and second transceivers enable the sensor module and the apparatus to communicate without being adversely affected by, or adversely affecting, the operation of the imaging scanner.

The invention also provides a method of wirelessly communicating data indicative of at least a condition of a patient exposed to a scanner of an MR system. The method involves acquiring the data from a sensor mechanism attached to the patient, and converting that data from optical format to electrical format. It further requires transmitting in radio frequency (RF) format the data received in electrical format, and then receiving the data transmitted in the transmitting step. The method further involves converting the data received in the receiving step from electrical format to optical format, and conveying the data to an apparatus remote from the patient. The method requires that the communication of data be accomplished without being adversely affected by, or adversely affecting, the operation of the MR system.

In a related aspect, the invention also provides a method of wirelessly communicating data in an imaging suite. The method includes the step of providing a first transceiver connected to a sensor for transmitting the data received therefrom and for conveying to the sensor the data transmitted thereto. It also involves the step of providing a second transceiver, which is connected to an apparatus remote from the first transceiver, for conveying the data received from the first transceiver to the apparatus and for transmitting to the first transceiver the data received from the apparatus. The method requires the first and second transceivers to communicate without being adversely affected by, or adversely affecting, the operation of equipment in the imaging suite.

In a presently preferred embodiment, the invention provides a communications module for wirelessly communicating electrocardiographic (ECG) signals obtained from a patient situated in a noisy environment. The module includes at least one RF filter, a lead select network, a differential amplifier, an amplifier circuit, a signal processing circuit, a modulator circuit, a transmitter circuit, and a filter circuit. The RF filter(s) is linked to a sensor of bioelectric signals for removing therefrom frequencies outside those carrying the bioelectric signals. The lead select network is used to select, in response to control signals, the appropriate lead(s) of a multiple-lead lead-set from which to pickup selected one(s) of the bioelectric signals. The differential amplifier is used to derive the ECG signals from the bioelectric signals selected via the network. The amplifier circuit is used to amplify the ECG signals received from the differential amplifier, and the signal processing circuit is used to improve the condition of the ECG signals received from the amplifier circuit. The modulator circuit digitally modulates a carrier signal in accordance with the ECG signals it receives from the signal processing circuit to form a modulated signal therewith. The transmitter circuit is connected to the modulator circuit for transmitting the modulated signal received therefrom. Connected to the transmitter circuit, the filter circuit allows the modulated signal to pass while effectively attenuating unwanted frequencies.

In a related embodiment, the invention also provides a communications module for wirelessly communicating physiologic signals obtained from a patient situated in a noisy environment. The module includes an input conditioning circuit, a signal processing circuit, a converter circuit, a transmitter circuit, and a filter circuit. The input conditioning circuit, which links to a sensor of the physiologic signals, is used to adapt the physiologic signals received therefrom for use in the module. The signal processing circuit improves the condition of the physiologic signals received from the input conditioning circuit, and the converter circuit converts the physiologic signals received from the signal processing circuit to digital signals corresponding thereto. The transmitter circuit is connected to the converter circuit and is used to transmit the digital signals received therefrom. The filter circuit is connected to the transmitter circuit for passing the digital signals and effectively attenuating unwanted frequencies.

### BRIEF DESCRIPTION OF THE DRAWINGS

The invention and its various embodiments will be better understood by reference to the detailed disclosure below and to the accompanying drawings, wherein:

FIGS. 1A, 1B and 1C illustrate the layout of an MR suite inclusive of the scanner room in which the scanner and patient table are located, the control room in which the computer console for controlling the scanner is situated, and the equipment room in which various control subsystems for the scanner are sited.

FIG. 2 is a first preferred embodiment of a system of communicating ECG data wirelessly between the patient and monitoring equipment located in the MR suite;

FIG. 3 is a second preferred embodiment of a system of communicating ECG data wirelessly between the patient and monitoring equipment located in the MR suite;

FIG. 4 is a block diagram for a preferred embodiment of the wireless ECG sensor module of the type shown in FIGS. 2 and 3;

FIG. 5 is a block diagram for a preferred embodiment of a wireless patient sensor module capable of communicating essentially any type of data between the patient and monitoring equipment located in the MR suite;

FIG. 6 is a schematic diagram of a transceiver assembly for converting the physiologic data obtained from a patient sensor module from optical signals to RF signals according to the second preferred embodiment of the present invention;

FIG. 7 is a schematic diagram of a transceiver assembly for converting the physiologic data received as RF signals back to optical signals according to the second preferred embodiment of the present invention; and

FIG. 8 is a picture of two transceivers, built according to the second preferred embodiment of the present invention, for communicating physiologic data between a sensor module on a patient from which the physiologic data is acquired and the monitoring equipment within the electromagnetic environment of the MR suite.

### DETAILED DESCRIPTION OF THE PRESENTLY PREFERRED AND ALTERNATIVE EMBODIMENTS OF THE INVENTION

Although the invention herein described and illustrated is presented primarily in the context of systems for and methods of wirelessly communicating physiologic data within and around the MR environment, the reader will understand that the invention can be applied or adapted not only to other types of data but also to a variety of other environments. Various embodiments and related aspects of the invention will now be described with reference to the accompanying drawings, in which like elements have been designated where possible by the same reference numerals.

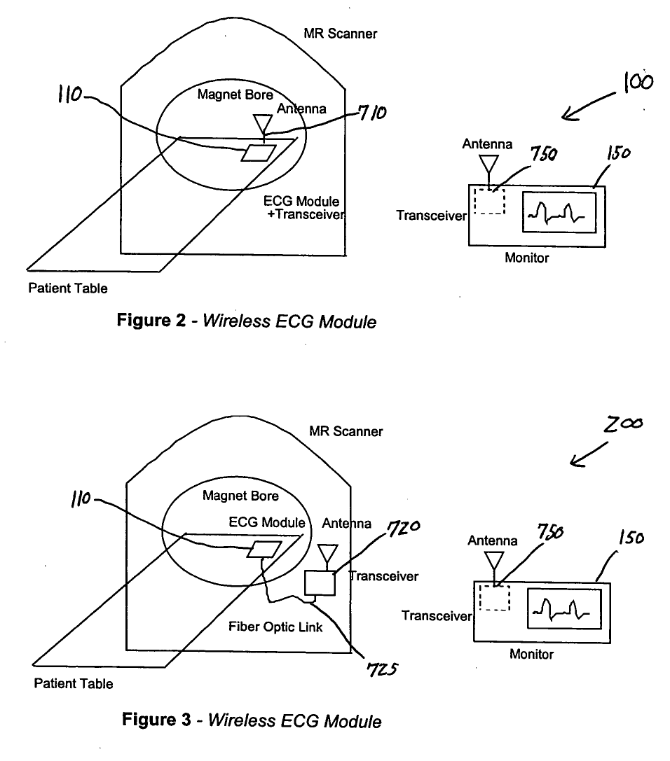

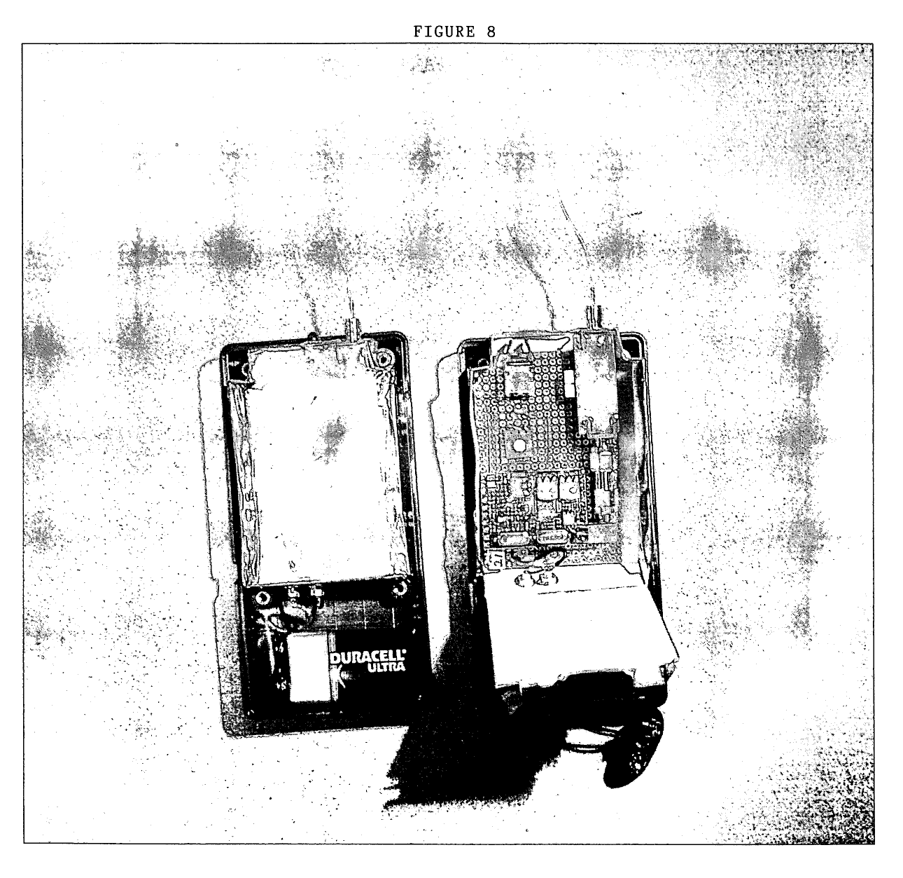

FIGS. 2-8 illustrate several embodiments of the invention, namely, systems, methods, and associated devices for wirelessly communicating physiologic signals or other data in an electromagnetically noisy environment. More specifically, these figures show systems that permit wireless bidirectional or unidirectional communication of data between a patient within the bore of an MR scanner and the monitoring equipment therefor located elsewhere in the MR suite. As will be apparent from the embodiments disclosed below, the invention is preferably implemented through RF communication techniques, but may also be carried out using optical communication schemes. RF communication schemes are preferred because line of sight restrictions are less of a problem. It is best to use RF signals in the microwave region to communicate the physiologic data out of the bore because the bore may effectively act as a waveguide for RF signals, with a cutoff frequency in the lower frequency ranges (<500 MHz).

FIG. 2 illustrates a first embodiment of a system, generally designated 100, for wireless communication between an ECG module 110 and a monitor 150 located elsewhere in the MR suite. The ECG module 110 and the monitor 150 each include a transceiver and associated antenna to enable communication, preferably bidirectional, between them. In this particular depiction, the ECG module 110 includes an integral transceiver and antenna assembly 710, as does monitor 150 with transceiver and antenna assembly 750. The design of the ECG module 110 and its transceiver assembly 710 permits use on the patient even when exposed to the electromagnetically noisy environment found within the bore of an MR scanner. As discussed in detail subsequently, this design therefore requires use of a communication scheme that not only ensures reliable communication between the ECG module 110 and monitor 150 but also avoids interfering with the MR system around which it is used.

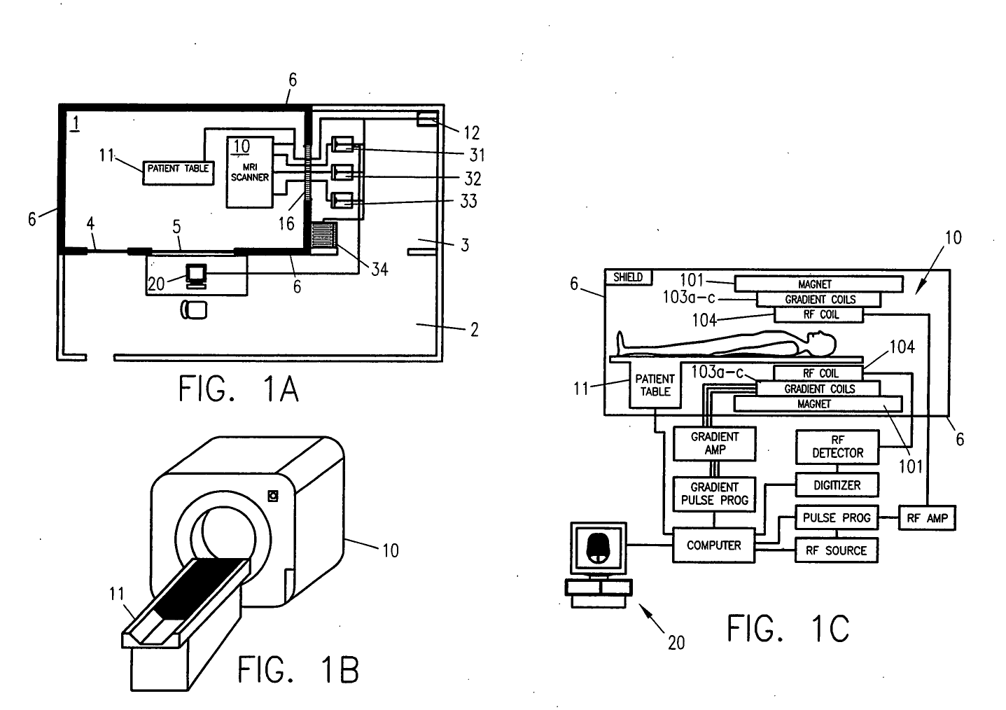

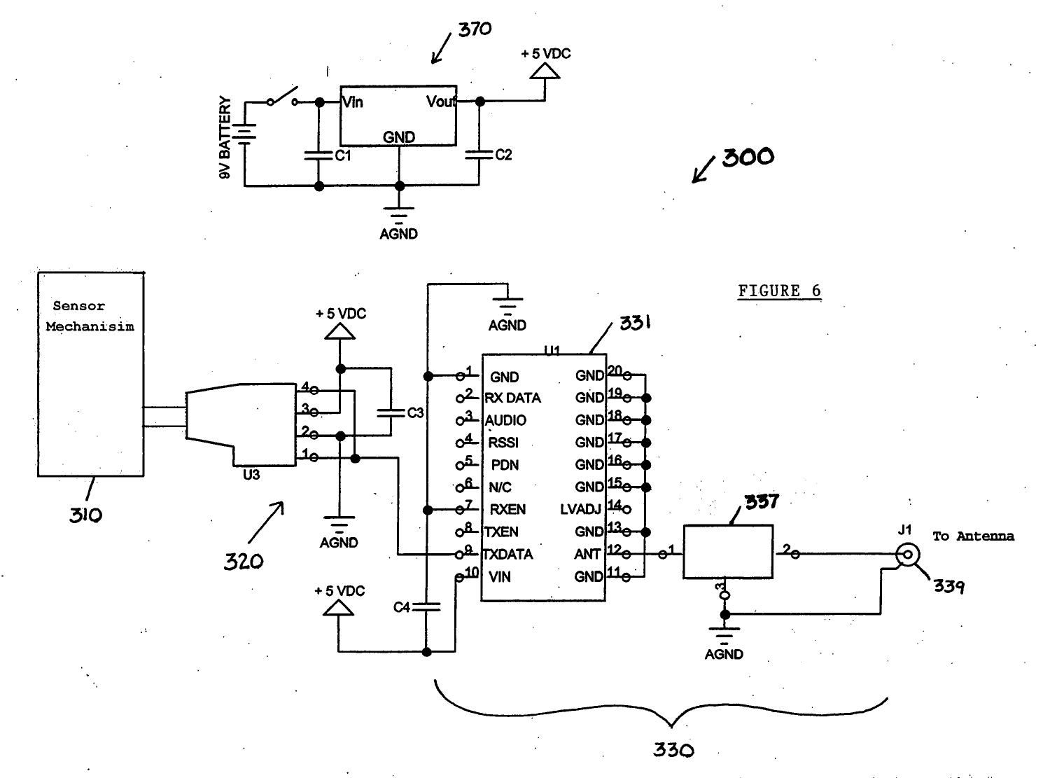

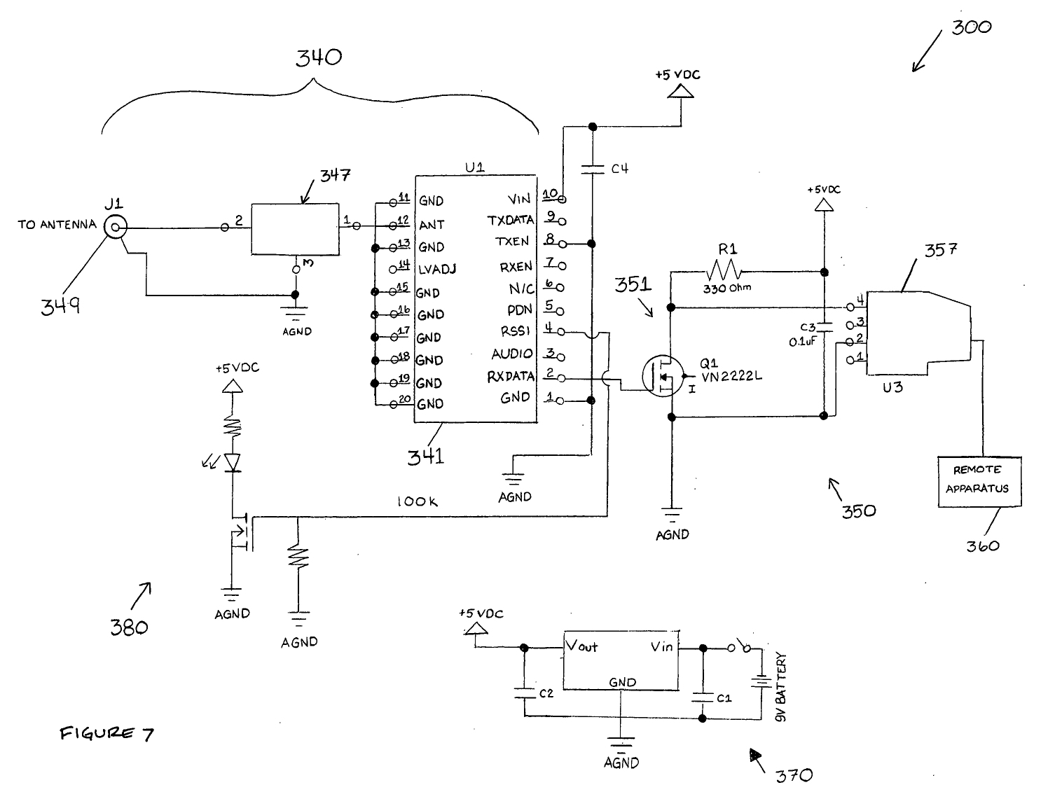

FIG. 3 shows a second embodiment of a system, generally designated 200, for wireless communication between ECG module 110 and the remotely located monitor 150. Although similar to the embodiment shown in FIG. 1, this embodiment employs a transceiver assembly 720 that is not integral to ECG module 110. More specifically, ECG module 110 and transceiver assembly 720 are connected by means of a fiber optic communications link 725 rather than the more direct connection contemplated by FIG. 2. The communications link 725 is preferably implemented using the communications scheme presented below in connection with FIGS. 6 and 7.

FIGS. 6 and 7 illustrate the circuit schematics for two transceiver assemblies capable of being used, or being adapted for use, in the embodiments of present invention. Together these transceiver assemblies serve as the central parts of a system that can be used to wirelessly communicate data between a sensor module on a patient and a remotely-located monitoring apparatus, or, more broadly, between any two devices of a bifurcated system. In a presently preferred embodiment, this system, generally designated 300, includes a sensor mechanism 310, a first transducer circuit 320, a first RF transceiver circuit 330, a second RF transceiver circuit 340, a second transducer circuit 350, and two power regulating circuits 370. One or both transceiver assemblies of system 300 may also optionally feature a link status indicator circuit 380.

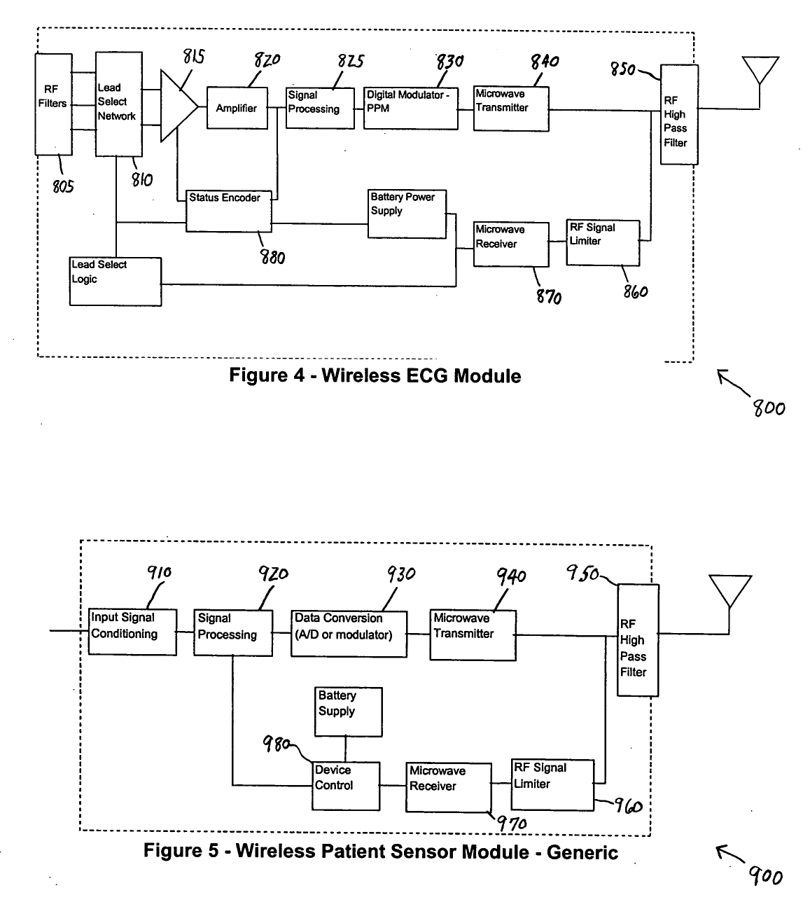

The sensor mechanism 310 of system 300 can take advantage of any of several prior art electrode/lead-set assemblies of the type used to conduct electrical currents from the surface of living tissues. Particularly when system 300 is to be used in noisy environments such as that found in a typical MR suite (e.g., sensor mechanism 310 within the bore of the scanner), provision should be made so that the bioelectric signals embodied in such electrical currents and carried by the lead(s) of the lead-set be accompanied by as little noise as possible. Although presented herein in the context of an ECG module designed for use in the MR suite, it should be apparent that sensor mechanism 310 can be also implemented in other forms such as an EEG module, an EMG module, or even an EOG module. However implemented, the sensor mechanism 310 is the device used to acquire data indicative of the condition of the patient. In a preferred manifestation, sensor mechanism 310 may also be rendered capable of conveying data pertaining to its operational status and, further, of acting upon control signals it receives. This data is intended to be communicated to the remotely-located monitoring apparatus 360 for the purpose of visual display, audio alert, or other suitable action. Two preferred implementations of a communications module, one specific to the ECG context and one generic, in which sensor mechanism 310 may be at least partly incorporated, are disclosed below in connection with FIGS. 4 and 5.

The first transducer circuit 320 is used to convert the data received from the sensor mechanism 310 from optical format to electrical format. This opto-electric transducer may take the form of the HFBR-2523 Fiber Optic (FO) Transceiver manufactured by Agilent Technologies, Inc. As disclosed in Agilent Publication 5988-1765EN, incorporated herein by reference, the HFBR-2523 FO Transceiver is capable of providing a high degree of immunity from sources of electromagnetic interference (EMI) and radiofrequency interference (RFI). Consequently, the HFBR-2523 Transceiver is well suited for use in noisy environments such as those found in MR suites. As shown in FIG. 6, pin 3 of the HFBR-2523 Transceiver connects to power regulator circuit 370 from which it receives 5V DC power, and pin 2 connects to ground. The HFBR-2523 Transceiver receives the optical data from sensor mechanism 310. The electrical data output by the HFBR-2523 FO Transceiver exits via pins 1 and 4, which provide the electrical data to the input of first RF transceiver circuit 330.

The first RF transceiver circuit 330 is connected to the first transducer circuit 320 and is used to transmit the data it receives therefrom. It includes a transceiver module 331, a filter 337 and an antenna 339. The transceiver module 331 can be implemented in the form of the TR-916-SC-PA RF Transceiver Module sold by Linx Technologies, Inc. As shown in FIG. 6, and disclosed in the SC-PA SERIES TRANSCEIVER MODULE DESIGN GUIDE published by Linx, incorporated herein by reference, TR-916-SC-PA module receives at its TXDATA terminal the electrical data signal output from pins 1 and 4 of the HFBR-2523 FO Transceiver. When switched to the transmit mode by applying high and low logic levels to the TXEN and RXEN terminals, respectively, and by biasing the PDN pin to open, the TR-916-SC-PA module transmits from its ANT pin the frequency modulated signal on which the data signal applied to the TXDATA pin is carried. The TR-916-SC-PA module 331 is capable of transmitting the modulated signal at a center frequency of 916.48 MHz with a data rate as high as 33.6 Kbps.

The filter 337 is preferably implemented as the TKS2606CT-ND Dielectric Filter made by Toko, Inc. As disclosed in Datasheet (T042) 749, incorporated herein by reference, the TKS2606CT-ND filter has a center frequency of 915 MHz and a bandwidth of +13.0 MHz. With its input connected to the ANT terminal of transceiver module 331, the filter 337 will effectively remove unwanted signals and spurious noise while allowing the modulated signal received thereat to pass to antenna 339. Although the TKS2606CT-ND model is a bandpass filter, even low pass, high pass and notch filters may be employed to attenuate frequencies outside those carrying the pertinent data.

The antenna 339 for the first RF transceiver circuit 330 can take the form of any number of commercially available antennas. One example of an acceptable antenna is the ANT-916-CW-QW antenna made by Linx Technologies, Inc. This type of antenna may also be employed for antenna 349 of the second RF transceiver circuit 340.

Configured to receive the data transmitted by first RF transceiver circuit 330 via antenna 339, the second RF transceiver circuit 340 includes a transceiver module 341, a filter 347 and an antenna 349. The modulated signal radiated by antenna 339 of first transceiver circuit 330 is initially received by antenna 349 and then passed to filter 347. Like its counterpart in the first transceiver circuit, filter 347 can be implemented with the TKS2606CT-ND bandpass filter or, alternatively, with a low pass, high pass or notch filter. The filtered modulated signal output by filter 347 is then conveyed to the ANT terminal of transceiver module 341.

Like transceiver module 331, transceiver module 341 can be implemented in the form of the TR-916-SC-PA RF Transceiver unit. When switched to the receive mode by applying low and high logic levels to the TXEN and RXEN terminals, respectively, and by opening the PDN terminal, the TR-916-SC-PA module is able to receive at its ANT pin the frequency modulated signal transmitted by first RF transceiver circuit 330. The TR-916-SC-PA module 341 then demodulates the modulated signal and conveys to the second transducer circuit 350 via its RXDATA terminal the resulting data signal.

The second transducer circuit 350 is used to convert the electrical data signal it receives from transceiver module 341 to optical format. It includes a driver circuit 351 and an electro-optic transducer 357. The driver circuit is recommended primarily to assure there is sufficient power to drive the electro-optic transducer 357, particularly if one anticipates using a long fiber optic cable to interconnect the transducer 357 and the remote apparatus 360. The driver circuit 351 can be manifested as an N-channel MOSFET such as the VN2222L chip disclosed in Document No. 70213 S-04279-Rev. F, published 16 Jul. 2001 by Vishay Intertechnology, Inc. The electro-optic transducer 357 can take the form of the HFBR-1523 FO Transceiver manufactured by Agilent Technologies, Inc, as disclosed in Agilent Publication 5988-1765EN. As shown in FIG. 7, the gate of the MOSFET receives the electrical data signals from the RXDATA terminal of transceiver module 341. The source and drain terminals of driver circuit 351 provide the amplified electrical output to terminals 2 and 4 of the HFBR-1523 FO Transceiver. The resulting optical data signals output by the HFBR-1523 FO Transceiver are then routed to the remote apparatus 360 via a fiber optic cable or other suitable waveguide.

The regulator stage 490 may be implemented using any one of a variety of regulator circuits known in the electrical/electronic arts. The regulator illustrated in FIG. 6A, for example, is a precision voltage reference produced and sold under Model No. REF02 by Analog Devices, Inc., of Norwood, Mass. As disclosed in Rev. C (2002) of its specification sheet, incorporated herein by reference, the REF02 regulator 490 is capable of providing a stable 5V DC output, regulated to approximately +i %, from the 15V DC input received from the GLM65-15 power supply 120 via DC power cord 130. This 5V DC reference voltage is supplied to both the output selector stage 410 and the indicator stage 480.

The power regulating circuit 370 for each of the transceiver assemblies in system 300 can take the form of any of a variety of regulator circuits known in the electrical/electronic arts. One such regulator is the LM7805 regulator produced by Fairchild Semiconductor Corp. As disclosed in the MC78XX/LM78XX/MC78XXA Datasheet published 2 Jul. 2001, incorporated herein by reference, the LM7805 regulator is capable of providing a stable 5V DC output from a 9V DC input. The first transceiver assembly has one regulating circuit 370 with which to provide a 5V DC reference voltage to both the HFBR-2523 FO Transceiver 320 and the TR-916-SC-PA transceiver module 331. Similarly, the other transceiver assembly has a regulating circuit 370 to provide a 5V DC reference voltage to the driver circuit 351, the HFBR-1523 FO Transceiver 357, and the TR-916-SC-PA transceiver module 341.

Preferably incorporated only into the second transceiver circuit 340, the link status indicator circuit 380 of system 300 may be implemented with an N-channel MOSFET, such as the VN2222L chip, and a light-emitting diode (LED). As shown in FIG. 7, the LED has its anode connected to the 5V DC voltage provided by regulating circuit 370, with its cathode connected to drain of the MOSFET. The gate of the MOSFET connects to the RSSI (i.e., “Received Signal Strength Indicator”) terminal of transceiver module 341 from which it receives biasing signals when module 341 is transmitting or receiving. When so biased at its gate, the MOSFET is turned on thus connecting its drain to source and thereby providing a path to ground to power the LED. The primary purpose of indicator circuit 380 is to provide a visual indication to the user when data is being transmitted between sensor mechanism 310 and remote apparatus 360.

The data communicated by sensor mechanism 310 need not be limited solely to physiologic data. It can also include data pertaining to the operation and status of the sensor mechanism 310 itself. Examples of the types of operational data that may be communicated include information as to (i) the state of charge of the battery that powers the regulating circuit 370 and, if applicable, (ii) which lead(s) of a multi-lead lead-set the underlying physiologic signals were obtained from.

Although the foregoing focuses on a unidirectional communication scheme, the system 300 is also capable of bidirectional communication. The two transducer circuits 320 and 350, the two TR-916-SC-PA transceiver modules 331 and 341, and the two filters 337 and 347 are all designed for two-way communication. Consequently, the present invention also enables the transmission of data from the remote apparatus 360 to sensor mechanism 310. Examples of the types of data that may be communicated back to sensor mechanism 310 include control signals. Such control signals could be used to command sensor mechanism 310 to select only certain lead(s) of a multiple-lead lead-set from which to pickup the underlying physiologic signals. In the ECG context, for example, in which a 3-lead lead-set is used, the control signals could direct sensor mechanism 310 to choose from those 3 leads the two from which to pickup bioelectric signals, and thus to transmit the ECG signal derived from those two bioelectric signals.

The present invention also contemplates a method of wirelessly communicating data, such as physiologic signals indicative of the condition of a patient who is exposed to the scanner of an MR system. In a presently preferred embodiment, the method involves acquiring the data from a sensor module (e.g., ECG module 110) attached to the patient, and converting that data from optical to electrical format. The electrical data signal is then transmitted in RF format by a first transceiver assembly associated with the sensor module. The method also includes the steps of using a second transceiver assembly remote from the sensor module to receive the RF data signal transmitted by the first transceiver assembly, and then converting that data signal from electrical to optical format. The optical data signal is then conveyed from the second transceiver assembly to a remotely-located apparatus (e.g., monitor 150) with which it is linked.

Furthermore, the method also preferably enables communication from the remote apparatus to the sensor module. In this presently preferred embodiment, this involves converting the data received from the remote apparatus from optical to electrical format, conveying that electrical data signal to the second transceiver assembly, and then transmitting it in RF format to the first receiver assembly. The next steps involve using the first transceiver assembly to receive the transmitted RF data signal, and then converting that data signal from electrical to optical format for conveyance to and use by the sensor module. Examples of the type of data that may be communicated back to sensor module include the aforementioned control signals described in connection with the preferred system embodiment. [[As disclosed more fully below, the communication between the sensor module and the remote apparatus must be accomplished without being adversely affected by, or adversely affecting, the operation of the MR system.]]

The present invention also provides two preferred implementations of a communications module—one specific to the ECG context and one generic—capable of communicating with the remotely-located monitoring apparatus 150/360. FIG. 4 illustrates a communications module adapted to an ECG electrode/lead-set assembly for wirelessly communicating ECG signals obtained from a patient situated in a noisy environment, such as that found within the bore of a scanner. In its preferred embodiment, this communications module, generally designated 800, includes an RF filter 805, a lead select network 810, a differential amplifier 815, an amplifier circuit 820, a signal processing circuit 825, a modulator circuit 830, a transmitter circuit 840, a filter circuit 850, and an antenna 855. The RF filter 805 is linked to an ECG electrode/lead-set sensor from which it receives a bioelectric signal from each lead. The filter is tuned so that frequencies outside those carrying the bioelectric signals are removed. The lead select network 810 is used to select, in response to control signals sent from remote apparatus 150/360, the particular lead(s) of the electrode/lead-set sensor from which to pickup bioelectric signals. The differential amplifier 815 derives the ECG signals from the bioelectric signals selected by the network 810. The amplifier circuit 820 is used to amplify the ECG signals received from the differential amplifier. The signal processing circuit 825 is preferably used to improve the condition of the ECG signals received from the amplifier circuit. The modulator circuit 830 digitally modulates a carrier signal in accordance with the ECG signals received from the signal processing circuit to form a modulated signal therewith. The transmitter circuit 840, preferably configured for transmitting in the microwave band, is connected to the modulator circuit for transmitting the modulated signal received therefrom. The filter circuit 850 passes the modulated signal received from the transmitter circuit yet attenuates extraneous noise and other unwanted frequencies. The modulated signal is then radiated by a suitable antenna.

To enable bidirectional communication, the communications module may also include a limiter circuit 860, a receiver circuit 870, and an encoder circuit 880. The limiter circuit 860 is linked to filter circuit 850 for limiting the amplitude of control signals picked up by the antenna from the remote apparatus 150/360. The receiver circuit 870 is connected to the limiter circuit from which it receives the control signals. The encoder circuit 880, optionally in response to the control signals, may be used to encode the outgoing ECG signals with information pertaining to various operational parameters. Examples of such parameters include information as to the amount of power available to the communications module and the particular lead(s) of the electrode/lead-set sensor from which the bioelectric signals were picked up.

FIG. 5 illustrates a communications module adapted to a more generic form of patient sensor, such as EEG module, an EMG module, or even an EOG module. This communications module 900 includes an input conditioning circuit 910, a signal processing circuit 920, a converter circuit 930, a transmitter circuit 940, a filter circuit 950, a limiter circuit 960, a receiver circuit 970, and a control circuit 980. This circuitry collectively performs largely the same functions as those described in connection with communications module 800, with appropriate accommodation made for the different type of patient sensor(s) with which it can be used.

In addition to the signal acquisition and processing circuitry, each of the communications modules 800 and 900 preferably include a means for assuring integrity of the communications between itself and the remote apparatus with which it communicates. For example, the communications module could employ CRC (cyclic redundancy checking) or like verification testing to determine the rate of error in the transmission of communications between itself and the remote apparatus.

In the disclosed embodiments, the communication of data must be accomplished without adversely affecting, or being adversely affected by, the operation of equipment in whose environment the communication occurs. When used in the MR suite, for example, the transceiver assemblies and communications modules disclosed herein must include provisions for reducing the potential for artifacts in the images from RF noise within the sensitive listening area of the electromagnetic frequency spectrum of the scanner (near the Larmor frequency). Otherwise, such noise could cause artifacts in the images obtained during a scanning procedure. The transceiver assemblies and communications modules must also be protected from the high energy RF signals radiated from the scanner when a scan is in progress. Microwave communications may be performed license free using the 915 MHz, 2.4 GHz, or 5.8 GHz Industrial, Scientific, and Medical (ISM) bands for communication. In addition, other licensed microwave frequency bands may be used, such as those discussed in U.S. Patent Application Publication 2003/0058502 A1 cited above. At these higher frequencies, smaller antennas may be used, which will beneficially reduce the RF signal energy received from the scanner due to the length of the antenna is <<λ/10 relative to the scanner wavelength used. As with any type of equipment placed in the bore of the scanner, non-magnetic materials should be used for construction of the antenna and all other electronics. Shielding should also be used to further reduce the susceptibility of the communications modules to the electromagnetic energy emanated by the scanner.

For communication schemes that use microwave frequencies, such as in the 2.4 GHz ISM band (e.g., 802.11b, Bluetooth™), the filter may be constructed as a microwave stripline filter, waveguide filter, surface acoustic wave (SAW) filter, or dielectric filter. (An example of a dielectric filter that works well at 2.4 GHz is from Toko, Inc., Model No. TFM1B-2450T-10. The device has a center frequency of 2.450 GHz with a pass band width of 50 MHz and a maximum pass band insertion loss of 2.3 dB). It is also possible to use the proposed Ultra Wide-band (UWB) techniques recently approved by the Federal Communications Commission (FCC). UWB radio systems typically employ pulse modulation whereby extremely narrow pulses are modulated and emitted to convey or receive information. The emission bandwidths generally exceed one Gigahertz. In some cases, “impulse” transmitters are employed where the pulses do not modulate a carrier. Instead, the radio frequency emissions generated by the pulses are applied to an antenna, the resonant frequency of which determines the center frequency of the radiated emission. The bandwidth characteristics of the antenna will act as a low-pass filter, further affecting the shape of the radiated signal.

The high frequency signals used for such communication are substantially above the Larmor frequency of the scanner, and are thus not likely to cause interference with or be affected by the MR system. A high pass filter with a cutoff frequency above the Larmor frequency, and sufficient stop band attenuation (e.g., 80 to 100 dB of signal loss), will allow the data signals to pass but reduce any lower frequency signals that could potentially interfere with the MR scan or create image artifacts.

To protect the electronics, the communication module 800/900 includes a limiter circuit 860/960 to block any excess RF energy coupled onto the antenna from the scanner. Devices such as PIN-PIN diode limiters or PIN-Schottky diode limiters are preferably used to block RF energy from the scanner coupled in by the antenna above some limit (typically ˜10 dBm), but allow sufficient energy to pass when transmitting the physiologic data. A microwave PIN diode is a current-controlled semiconductor device that acts as a variable resistor at RF and microwave frequencies. When a device is used as a shunt across an antenna input, it can effectively limit input signals when they become excessive. A combination of two PIN diodes can be used to provide receiver input protection and as an antenna transmit receive switch (i.e., they can be used in a circuit that will isolate the receiver from the transmitter when the transmitter is active.)

During a scanning procedure, it may also be possible to transmit the data signals from the communication modules in the brief time periods when the RF signals or gradient coils of the scanner are inactive. This allows for more noise free and reliable communication. For ECG applications, detection of these “off-time” windows for the scanner can be performed by monitoring the leads of the electrode/lead-set assembly for the characteristic signatures of these RF and gradient induced signals from the scanner.

For the system 100 illustrated in FIG. 2, the ECG module 110 connects via a fiber optic link to transceiver assembly 710, which may be situated either near or on the patient table or on the face of the scanner housing. This approach allows the use of non-microwave RF signals for communication. In addition, this approach allows for greater flexibility in placement of the monitoring equipment (e.g., display), because non-microwave RF signals are not as directional. This method also offers the advantage of the possibility to use a separate larger battery power source for the transceiver, allowing communication over greater distances and longer operating time for the system. Note that it is possible to replace an existing fiber optic or wired connection with a wireless connection by converting the optical or electrical signals to RF signals through some modulation means. Pulse position modulation or other high power efficiency modulation schemes are preferable for battery-operated equipment.

For maximum flexibility of equipment placement, the antennas herein disclosed are preferably circularly polarized, such as by using a spiral or helical antenna design. While potentially losing a nominal 3 dB of gain for each antenna, this allows for more flexibility in the orientation, polarization, and placement of the antennas on the communicating devices. If the transceiver assemblies/communications modules are likely to be in a fixed location within the operating environment, antenna designs with greater gain/directivity, such as parabolic, horn or Yagi antenna designs, may be used to optimize signal strength coupling and system signal to noise ratio (SNR).

In addition, it is possible to use broadband antennas or antennas that operate at frequency multiples to allow for communication at several frequencies. Spiral antenna designs, for example, are naturally broadband and could be used to operate at more than one frequency range. Multiple antennas may also be useful for antenna diversity as a way to deal with the effects of multipath signal transmission, especially in the scanner room, which is likely to be a highly reflective environment due to the metal shielding and the equipment typically located therein. It is preferable to place any directive antennas for increased signal gain in the control room, where multipath effects are likely to be less than those in the scanner room.

In the case of transceiver assemblies 710/720 and communications module 800, it is possible to use the lead(s) of the electrode/lead-set assembly as the antenna. The antenna may be implemented as additional conductors with the lead-set. Alternatively, the wires that are part of the lead-set may be used. Appropriate bandstop filtering must be included to eliminate entry of RF energy from the scanner, but to allow exit of higher frequency signals for RF communication. If the lead-set assembly is used as the antenna, the lead(s) must be of the appropriate length and must be properly tuned to make sure that they are efficient antennas. Also, the RF transmit power from the transceiver assembly/communications module must be limited to safe levels.

Because the transceiver assemblies/communications modules are battery powered, some means of power management is useful to help preserve operating time for these devices. This can be done in a number of ways. First, the transceiver assembly/module can be rendered capable of monitoring when data signals are being received from the remotely-located transceiver. When the data signals are received, the rest of the system can be powered up. If a signal from the device located outside of the bore of the scanner is not present for some time period, the rest of the system can be powered down. Second, the transceiver assembly/module can monitor for motion, and power up for some time period after motion is detected. Finally, the transceiver assembly/module can monitor for RF energy from the scanner, indicating that scanner activity is taking place, and power up for some time period. Also, as part of power management, the transceiver assembly/module can transmit low battery warnings or transmit data at a reduced rate to indicate that the battery is low, while extending the remaining battery operating period.

The wireless link disclosed herein can also be adapted for uses other than the monitoring of physiologic data. For example, the wireless techniques used could also be applied to control an infusion device connected to the patient. For example, it could be used to program, start, and stop an infusion as well as to communicate infusion status to another device. Another potential application is the control of adjustable body coils for MRI, such as the head and neck coil used for measurement of the temporomandibular joint (TMJ). The concepts of the present invention can also be applied to bifurcated systems of the type used to monitor the reaction of patients during functional MRI studies. Similarly, the invention is also equally applicable to systems capable of providing wireless video and/or sound to and from the patient (e.g., headphones or video devices).

The presently preferred and alternative embodiments for carrying out the invention have been set forth in detail according to the Patent Act. Persons of ordinary skill in the art to which this invention pertains may nevertheless recognize alternative ways of practicing the invention without departing from the spirit of the following claims. Consequently, all changes and variations that fall within the literal meaning, and range of equivalency, of the claims are to be embraced within their scope. Persons of such skill will also recognize that the scope of the invention is indicated by the claims below rather than by any particular example or embodiment discussed or shown in the foregoing description.

Accordingly, to promote the progress of science and useful arts, I secure by Letters Patent exclusive rights to all subject matter embraced by the following claims for the time prescribed by the Patent Act.

1. A system for wirelessly communicating physiologic data indicative of a condition of a patient exposed to a scanner of a magnetic resonance (MR) system, said system comprising:
(a) a sensor mechanism for acquiring from said patient said physiologic data;
(b) a first transducer circuit connected to said sensor mechanism for converting said physiologic data received therefrom from optical format to electrical format;
(c) a first RF transceiver circuit connected to said first transducer circuit for transmitting said physiologic data received therefrom;
(d) a second RF transceiver circuit remote from said first RF transceiver circuit for receiving said physiologic data transmitted by said first RF transceiver circuit; and
(e) a second transducer circuit connected to said second RF transceiver circuit for converting said physiologic data received therefrom from electrical format to optical format and for conveying said physiologic data to an apparatus remote from said sensor mechanism;
wherein communication between said sensor mechanism and said apparatus via said first and said second RF transceiver circuits is accomplished without adversely affecting, or being adversely affected by, operation of said MR system.

2. The system of claim 1 wherein said first RF transceiver circuit includes:
(a) an RF transceiver module having an input into which said physiological data from said first transducer circuit is received and an output from which said physiologic data is transmitted in radio frequency (RF) format;
(b) a filter connected to said output of said RF transceiver module for passing said physiologic data but effectively attenuating frequencies outside those carrying said physiologic data; and
(c) an antenna connected to said filter for radiating said physiologic data received therefrom.

3. The system of claim 2 wherein said filter is one of a bandpass filter, a high pass filter and a notch filter.

4. The system of claim 1 wherein said second RF transceiver circuit includes:
(a) an antenna for receiving said physiologic data transmitted by said first RF transceiver circuit;
(b) a filter connected to said antenna for passing said physiologic data but effectively attenuating frequencies outside those carrying said physiologic data; and
(c) an RF transceiver module having an input into which said physiological data from said filter is received and an output from which said physiologic data is conveyed to said second transducer circuit.

5. The system of claim 4 wherein said filter is one of a bandpass filter, a high pass filter and a notch filter.

6. The system of claim 1 wherein said second transducer circuit includes:
(a) a driver circuit having an input connected to an output of said second RF transceiver circuit; and
(b) an electro-optic transducer connected to an output of said driver circuit for converting said physiologic data received therefrom from electrical format to optical format and for conveying said physiologic data to said apparatus remote from said sensor mechanism.

7. The system of claim 1 wherein said sensor mechanism is an electrocardiographic (ECG) module for acquiring said physiologic data in the form of cardiac signals.

8. A system for wirelessly communicating data in an electromagnetically noisy environment, said system comprising:
(a) a first transducer circuit connected to a first device of a bifurcated system for converting said data received therefrom from optical format to electrical format;
(b) a first RF transceiver circuit connected to said first transducer circuit for transmitting said data received therefrom;
(c) a second RF transceiver circuit remote from said first RF transceiver circuit for receiving said data transmitted by said first RF transceiver circuit; and
(d) a second transducer circuit connected to said second RF transceiver circuit for converting said data received therefrom from electrical format to optical format and for conveying said data to a second device of said bifurcated system;
wherein a scheme of communication employed by said first and said second RF transceivers enables said first and said second devices to communicate without being adversely affected by noise in said environment.

9. The system of claim 8 wherein said first device of said bifurcated system includes an electrocardiographic (ECG) module for acquiring from a patient said data in the form of cardiac signals.

10. The system of claim 8 wherein said first device of said bifurcated system includes a sensor such that said data acquired thereby is indicative of a condition of a patient.

11. The system of claim 8 wherein said second device of said bifurcated system includes a monitoring apparatus capable of communicating with said first device via said second and said first RF transceiver circuits.

12. A system for wirelessly communicating data in a magnetic resonance (MR) suite, said system comprising:
(a) a first transceiver circuit connected to a sensor module for transmitting said data received therefrom and for conveying to said sensor module said data transmitted thereto; and
(b) a second transceiver circuit, connected to a monitoring apparatus, for conveying said data received from said first transceiver circuit to said monitoring apparatus and for transmitting to said first transceiver circuit said data received from said monitoring apparatus;
wherein said first and said second transceiver circuits communicate using predetermined frequencies outside a range of, and without adversely affecting, operation of equipment situated in said MR suite.

13. The system of claim 12 wherein said first transceiver circuit includes:
(a) a transceiver module having an input to which said data from said sensor module is conveyed and an output from which said data is transmitted in radio frequency (RF) format;
(b) a filter connected to said output of said transceiver module for passing said data but effectively attenuating frequencies outside those carrying said data; and
(c) an antenna connected to said filter for radiating said data received therefrom.

14. The system of claim 12 wherein said second transceiver circuit includes:
(a) an antenna for receiving said data transmitted by said first transceiver circuit in radio frequency (RF) format;
(b) a filter connected to said antenna for passing said data but effectively attenuating frequencies outside those carrying said data; and
(c) a transceiver module having an input into which said data from said filter is received and an output from which said data is conveyed to said monitoring apparatus.

15. The system of claim 12 wherein said data conveyed by said sensor module to said first transceiver circuit includes at least one of (i) physiologic signals indicative of a condition of a patient and (ii) operational signals indicative of a status of said sensor module.

16. The system of claim 15 wherein said data conveyed by said monitoring apparatus to said second transceiver circuit includes control signals commanding said sensor module to select appropriate lead(s) of a multiple-lead lead-set from which to pickup said physiologic signals.

17. The system of claim 12 wherein said data conveyed by said sensor module to said first transceiver circuit includes at least one of (i) cardiac signals indicative of heart condition and (ii) operational signals indicative of a status of said sensor module.

18. The system of claim 17 wherein said data conveyed by said monitoring apparatus to said second transceiver circuit includes control signals commanding said sensor module to select appropriate lead(s) of a multiple-lead lead-set from which to derive said cardiac signals.

19. A system for wirelessly communicating data obtained from a sensor module attached to a patient situated within an imaging scanner, said system comprising:
(a) a first transceiver linked to said sensor module for transmitting said data received therefrom; and
(b) a second transceiver, connected to an apparatus remote from said first transceiver, for conveying to said apparatus said data received from said first transceiver;
wherein said first and said second transceivers enable said sensor module and said apparatus to communicate without being adversely affected by, or adversely affecting, an operation of said imaging scanner.

20. The system of claim 19 further including a first transducer circuit between said sensor module and said first transceiver for converting said data received in optical format from said sensor module to electrical format for use by said first transceiver.

21. The system of claim 19 further including a second transducer circuit between said second transceiver and said apparatus for converting said data received in electrical format from said second transceiver to a format usable by said apparatus.

22. The system of claim 19 wherein said sensor module is an electrocardiographic (ECG) module for acquiring said data in the form of cardiac signals.

23. A method of wirelessly communicating data indicative of at least a condition of a patient exposed to a scanner of a magnetic resonance (MR) system, said method comprising the steps of:
(a) acquiring said data from a sensor mechanism attached to said patient;
(b) converting said data obtained from said patient from optical format to electrical format;
(c) transmitting in radio frequency (RF) format said data received in electrical format;
(d) receiving said data transmitted in said transmitting step;
(e) converting said data received in said receiving step from electrical format to optical format; and
(f) conveying said data to an apparatus remote from said patient;
wherein communication of said data is accomplished without being adversely affected by, or adversely affecting, an operation of said MR system.

24. The method of claim 23 wherein the step of acquiring said data includes using an electrocardiographic (ECG) module for acquiring said data in the form of cardiac signals.

25. A method of wirelessly communicating data in an imaging suite, said method comprising the steps of:
(a) providing a first transceiver connected to a sensor for transmitting said data received therefrom and for conveying to said sensor said data transmitted thereto; and
(b) providing a second transceiver, connected to an apparatus remote from said first transceiver, for conveying said data received from said first transceiver to said apparatus and for transmitting to said first transceiver said data received from said apparatus;
wherein said first and said second transceivers communicate without being adversely affected by, or adversely affecting, an operation of equipment in said imaging suite.

26. The method of claim 25 further including the step of providing a first transducer circuit between said sensor and said first transceiver for converting said data received (i) in optical format from said sensor to electrical format for use by said first transceiver and (ii) in electrical format from said first transceiver to optical format for use by said sensor.

27. The method of claim 25 further including the step of providing a second transducer circuit between said second transceiver and said apparatus for converting said data received (i) in electrical format from said second transceiver to a format usable by said apparatus and (ii) in optical format from said apparatus to electrical format for use by said second transceiver.

28. The method of claim 25 wherein said sensor is an electrocardiographic (ECG) module for acquiring said data in the form of cardiac signals.

29. A communications module for wirelessly communicating electrocardiographic (ECG) signals obtained from a patient situated in a noisy environment, said module comprising:
(a) at least one RF filter linked to a sensor of bioelectric signals for removing therefrom frequencies outside those carrying said bioelectric signals;
(b) a network for selecting, in response to control signals, appropriate lead(s) of a multiple-lead lead-set from which to pickup selected one(s) of said bioelectric signals;
(c) a differential amplifier for deriving said ECG signals from said bioelectric signals selected via said network;
(d) an amplifier circuit for amplifying said ECG signals received from said differential amplifier;
(e) a signal processing circuit for improving a condition of said ECG signals received from said amplifier circuit;
(f) a modulator circuit for digitally modulating a carrier signal in accordance with said ECG signals received from said signal processing circuit to form a modulated signal therewith;
(g) a transmitter circuit connected to said modulator circuit for transmitting said modulated signal received therefrom; and
(h) a filter circuit connected to said transmitter circuit for passing, and effectively attenuating frequencies outside of, said modulated signal.

30. The communications module of claim 29 wherein said transmitter circuit transmits said modulated signal at frequencies in the microwave band.

31. The communications module of claim 29 further including:
(a) a limiter circuit linked to said filter circuit for limiting an amplitude of control signals picked up by an antenna from a remote apparatus;
(b) a receiver circuit connected to said limiter circuit for receiving said control signals; and
(c) an encoder circuit for encoding said ECG signals with information pertaining to at least one of (i) an amount of power available to said communications module and (ii) from which of said leads of said multiple-lead lead-set were said ECG signals derived.

32. The communications module of claim 29 further including a means for assuring integrity of communications between said communications module and a remote apparatus with which said communications module communicates.

33. A communications module for wirelessly communicating physiologic signals obtained from a patient situated in a noisy environment, said module comprising:
(a) an input conditioning circuit linked to a sensor of said physiologic signals for adapting said physiologic signals received therefrom for use in said module;
(b) a signal processing circuit for improving a condition of said physiologic signals received from said input conditioning circuit;
(c) a converter circuit for converting said physiologic signals received from said signal processing circuit to digital signals corresponding thereto;
(d) a transmitter circuit connected to said converter circuit for transmitting said digital signals received therefrom; and
(e) a filter circuit connected to said transmitter circuit for passing, and effectively attenuating frequencies outside of, said digital signals.

34. The communications module of claim 33 wherein said converter circuit includes a modulator for digitally modulating a carrier signal in accordance with said physiologic signals received from said signal processing circuit to form said digital signals therewith.

35. The communications module of claim 33 wherein said transmitter circuit transmits said digital signals at frequencies in the microwave band.

36. The communications module of claim 33 further including:
(a) a limiter circuit linked to said filter circuit for limiting an amplitude of control signals picked up by an antenna from a remote apparatus;
(b) a receiver circuit connected to said limiter circuit for receiving said control signals; and
(c) a control circuit for controlling operation of said communications module in accordance with said control signals received from said remote apparatus.

37. The communications module of claim 33 wherein said control circuit enables said physiologic signals to be encoded with information pertaining to at least an amount of power available to said communications module.

38. The communications module of claim 33 further including a means for assuring integrity of communications between said communications module and a remote apparatus with which said communications module communicates.
